SEQUENCE LISTING (22 sequences)

SEQ ID NO: 1 - DNA, 21 bp, Artificial Sequence
  synthesized
tggtacagtctgggcaaggtt

SEQ ID NO: 2 - DNA, 21 bp, Artificial Sequence
  synthesized
cagcaactttgcucagaggtt

SEQ ID NO: 3 - DNA, 21 bp, Artificial Sequence
  primer
cagucgcguuugcgacuggtt

SEQ ID NO: 4 - DNA, 26 bp, Artificial Sequence
  synthesized
gactacgagatccacttgtttaagga

SEQ ID NO: 5 - DNA, 20 bp, Artificial Sequence
  primer
ctcacaaagccgatgaacca

SEQ ID NO: 6 - DNA, 20 bp, Artificial Sequence
  primer
atggcacaggccgcgtgagg

SEQ ID NO: 7 - DNA, 22 bp, Artificial Sequence
  primer
atccccgaggaatatgaaggac

SEQ ID NO: 8 - DNA, 22 bp, Artificial Sequence
  primer
gctcttccttgggtttgaagtg

SEQ ID NO: 9 - DNA, 26 bp, Artificial Sequence
  primer
ttccccacagatgacatcagcctcaa

SEQ ID NO: 10 - DNA, 17 bp, Artificial Sequence
  primer
ggcccgaccgatgaaag

SEQ ID NO: 11 - DNA, 20 bp, Artificial Sequence
  primer
gccaatgaacgaaccatcct

SEQ ID NO: 12 - DNA, 28 bp, Artificial Sequence
  primer
tcggcgagtacaggtccctggagagtga

SEQ ID NO: 13 - protein, 10 aa, Homo sapiens
VGEEDDGEYR

SEQ ID NO: 14 - DNA, 60 bp, Homo sapiens
cagatccccgaggaatatgaaggacaccatgtgatggagccacctgtcatcacggaacag

SEQ ID NO: 15 - DNA, 60 bp, Homo sapiens
gagaccttcggcgagtacaggtccctggagagtgacaacgaggagaaggcctttggcagc

SEQ ID NO: 16 - DNA, 21 bp, Artificial Sequence
  synthesized
ugguacagucugggcaaggtt

SEQ ID NO: 17 - DNA, 21 bp, Artificial Sequence
  synthesized
ccuugcccagacuguaccatt

SEQ ID NO: 18 - DNA, 21 bp, Artificial Sequence
  synthesized
cagcaacuuugcucagaggtt

SEQ ID NO: 19 - DNA, 21 bp, Artificial Sequence
  synthesized
cagcaacuuugcucagaggtt

SEQ ID NO: 20 - DNA, 21 bp, Artificial Sequence
  synthesized
ccucugagcaaaguugcugtt

SEQ ID NO: 21 - DNA, 21 bp, Artificial Sequence
  synthesized
gaaagguuccagggugacctt

SEQ ID NO: 22 - DNA, 21 bp, Artificial Sequence
  synthesized
ggucacccuggaaccuuuctt

(19) United States
(12) Patent Application Publication
(10) Pub. No.: US 2006/0127919 A1
(43) Pub. Date: Jun. 15, 2006

(54) Compositions and methods relating to cell adhesion molecule L1

(76) Inventors: Arie Abo, Oakland, CA (US); David A. Suhy, Castro Valley, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 11/067,029

(22) Filed: Feb. 25, 2005

Related U.S. Application Data
(60) Provisional application No. 60/547,835, filed on Feb. 25, 2004.

(51) Int. Cl. C12Q 1/68; G01N 33/574; C12P 19/34

(52) U.S. Cl. 435/6; 435/7.23; 435/91.2

(57) ABSTRACT

A method of identifying a compound that induces apoptosis in a cell is disclosed. The method includes contacting the cell with a putative apoptosis-inducing compound and determining whether the compound inhibits L1. Also disclosed are methods for inducing apoptosis in a cell by inhibiting L1. The invention further includes methods for the diagnosis of a tumor that include determining the level of L1 as a marker in a patient sample, the level of the marker being indicative of the presence of tumor cells.

### CROSS-REFERENCE TO RELATED APPLICATIONS

This application claims the benefit under 35 U.S.C. § 119, of U.S. Provisional Patent Application Ser. No. 60/547,935, entitled “Compositions and Methods Relating to Cell Adhesion Molecule L1,” filed Feb. 25, 2004, and incorporated by reference herein in its entirety.

### FIELD OF THE INVENTION

The invention relates to nucleic acid molecules and polypeptides identified as having a functional role in apoptosis. The invention also relates to methods for using the nucleic acid molecules and polypeptides of the invention, for example, as biomarkers, therapeutics and targets for therapeutics.

### BACKGROUND OF THE INVENTION

During early neuronal development, the processes of axon guidance and cell migration are regulated by proteins that mediate intercellular and cell-matrix interactions. The majority of these molecules fall into 3 distinct families: cadherins, integrins and the immunoglobulin (Ig) superfamily. The L1 cell adhesion molecule (referred to as L1CAM, L1-NCAM and CD171) is a type I membrane glycoprotein of the Ig superfamily that plays a role in promoting and directing axon growth during development of the nervous system (Seilheimer and Schachner, 1988; Draza and Lemmon, 1990). Specific structural elements define the L1 subfamily: each member contains six Ig-like domains at the amino-terminus, followed by either four or five fibronectin type III-like domains, a plasma membrane spanning region, and a highly conserved cytoplasmic tail (Moos et al., 1988; Kobayashi et al., 1991; Hlavin and Lemmon, 1991). Initially translated as a 140 kDa protein, L1 is post-translationally modified to produce a mature 200-220 kDa molecule when isolated from the cell surface (Patel et al., 1991).

Other known members of the L1 subfamily in mammalian systems include neurofascin, neuron-glial cell adhesion molecule (NgCAM), an NgCAM related cell adhesion molecule (NrCAM), and the close homologue of L1 (CHL1) (Davis et al., 1994; Holm et al., 1996). Similar in structure, it is believed that these molecules perform similar types of functions during embryogenesis. Specifically, L1 has been shown to have a function in inter-neuron adhesion, neurite fasciculation, synaptogenesis, as well as neurite outgrowth and migration (Fogel et al., 2003a). Indeed, mutations in the human L1 gene have been noted to cause CRASH (corpus collusum hypoplasia, retardation, adducted thumbs, spastic paraplegia, and hydroencephalus), a severe neurological X-linked syndrome estimated to occur in roughly one in 25,000 male births (Halliday et al., 1986). Additionally, CD171-knock out mice have pathologies that resemble those found within humans diagnosed with CRASH (Kamiguchi et al., 1998b) and targeted disruptions of L1 result in physical defects of the corticospinal tract and was found to result in dysfunctional axon guidance within this tissue. (Dahme et al., 1997; Cohen et al., 1998).

Although L1 was initially noted for its strong expression pattern in post-mitotic neurons and neural-derived tissues, moderate levels of the protein have been observed in other tissues. Several splice forms of L1 exist and it has been suggested that the different tissues may possess different isoforms of L1 (Reid et al., 1992; Jouet et al., 1995; Takeda et al., 1996; Itoh et al., 2000; Jacob et al., 2002). Among the most common variants, isoforms containing exons 2 and 27 were previously described in neuronal-based cells but were absent in other L1-expressing cells (Takeda et al., 1996). It has been postulated that these exons contribute to necessary functions of the protein within these tissues. For example, it has been shown that exon 27 is required for targeting of neuronal L1 to the axonal growth cone (Kamiguchi et al., 1998). Deletions of exon 2 have been associated with an in vitro reduction in neurite outgrowth promoting activity of L1 (Jacob et al., 2002) or have been linked with a subset of patients exhibiting symptoms of CRASH syndrome (Jouet et al., 1995).

While the apparent role of L1 in axon guidance justifies its expression pattern in neural tissues, the diverse distribution pattern of L1 expression noted in healthy and diseased tissues outside of the central nervous system suggests other functional roles for the protein. For instance, L1 protein was detected in lymphoid and myelomonocytic cells (Kowtiz et al., 1992, Kowtiz et al., 1993), including CD4+ T-cells (Ebeling et al., 1996) where it has been suggested that L1 may have endogenous function as a co-stimulatory molecule for T cell activation (Balaian et al., 2000). In studying the development of several organs by branching morphogenesis, expression of L1 was also found in kidney tissues (Deibic et al., 1998). Furthermore, treatment with anti-L1 antibodies caused renal defects in an organotypic culture model system indicating that the protein likely has an indispensable role in kidney development (Deibic et al., 1998). Yet, some of the strongest levels of L1 antigen expression have been noted in diseased human tissues including neuroblastomas (Figarella-Branger et al., 1990), carcinomas from renal (Meli et al., 1999) and lung tissues (Mayall et al., 1991; Miyahara et al., 2001) as well as in monocytic leukemia cells (Ebeling et al., 1996). L1 protein levels were also found to be significantly elevated in a large number of melanomas (Gabrielson et al., 1988; Fogel et al., 2003). A detailed statistical analysis over a broad range of human malignant melanomas showed a significant correlation between L1 overexpression and metastasis suggesting a functional role for L1 in the spread of the lesions (Theis et al., 2002). Further evidence of L1 function in metastasis was provided from in vitro studies demonstrating inhibition of melanoma cell migration by polyclonal L1 antibodies (Voura et al., 2001).

Perturbation of endogenous L1 function may significantly alter the growth characteristics of cells that express the protein. Neurite outgrowth of mouse and chick neurons on a strata coated with L1 were inhibited by Fabs against the L1 protein (Lemmon et al., 1989). Furthermore, It was previously demonstrated that neurite outgrowth of PC12 cells was inhibited in a concentration-dependent manner by a polyclonal antibody pool against L1 (Hall et al., 2000) or by antibodies directed against individual Ig-domains of the L1 protein (Yip et al., 2001). Additionally, disruption of the binding domain of functional heterophilic binding partners of L1 has also shown to interfere with L1-induced neurite outgrowth (Kristiansen et al., 1999).

Genetic Suppressor Elements (GSEs) are short, biologically active cDNA fragments that interfere with the function of the gene from which they are derived. GSEs act either as antisense RNA molecules against the full length cognate mRNA or as a transdominant peptide fragment. Libraries of random fragmented cDNA libraries or individually fragmented cDNA clones are introduced into cells via retroviral infection and are screened for the ability to generate a selectable phenotype. Selected GSEs are recovered and are sequenced; identification of the corresponding genes from which the GSEs were derived directly identifies target genes for the development of therapeutics. Examples of productive GSE-based screens include the identification of cellular and viral genes which have the ability to inhibit the human immunodeficiency virus (Dunn et al., 2004), the isolation of suppressor peptide fragments from the p53 protein (Mittleman et al., 1999), and utilization of the method for dissecting the functional domains of individual proteins such as the melanoma cell adhesion molecule (Satyamoorthy et al., 2001).

Recently, a GSE screen conducted in order to identify genes involved in eliciting an apoptotic response resulted in the isolation and identification of several hundred candidate genes. The details and results of that GSE screen are described in United States Patent Application Publication No. 2004/0170989 A1 entitled “Cellular Gene Targets For Controlling Cell Growth” and U.S. Provisional Patent Application No. 60/539,167, entitled “Cellular Gene Targets For Controlling Cell Growth,” each of which is incorporated by reference herein in its entirety. GSEs against L1 were also isolated from an independently performed screen in which the phenotypic selection criteria was based upon the ability of GSEs to inhibit cellular proliferation (Primiano et al., 2003).

There is an ongoing need to identify new targets and develop new assays for the identification of therapeutic compounds useful in the control of cell growth and tumor formation. In the present invention, a validation of the individual GSE elements against L1 was performed as well as an analysis of the expression of L1 protein in a panel of non-neuronal cell lines and L1 protein and mRNA expression in a panel of tumors cell lines.

### DESCRIPTION OF THE FIGURES

FIG. 1a-d shows apoptotic effects of GSE and small interfering ribonucleic acid (siRNA) species against L1-NCAM in HCT116 cells. FIG. 1(a) shows induction of apoptosis in a stable cell line of HCT116 cells containing an inducible GSE against L1 as assessed by FACS analysis of active caspase-3 levels. Bars represent the percentage of cells stained positive for the caspase protein relative to background levels of isotype control stained cells. FIG. 1(b) shows induction of apoptosis in HCT116 cells transfected with an siRNA species against L1 or an unrelated non-specific control duplex as assessed by FACS analysis of active caspase-3 levels. FIG. 1(c) shows determination of siRNA specificity by monitoring levels of surface L1 after transfection. Histograms of L1 treated cells (heaviest weighted line) show a decrease of L1 levels relative to cells treated with the unrelated non-specific control siRNA (medium weighted line) The histogram of isotype control staining is shown by the lightest weight line.

FIG. 2a-c shows an assessment of L1 RNA and surface protein expression levels in a variety of cell lines. FIG. 2(a) shows levels of surface L1 protein expression (heavier weighted line) as monitored by FACS analysis. The histogram of isotype control staining is shown by the lightest weight line. FIG. 2(b) shows LC-MS spectroscopy analysis of enriched preparations of plasma membrane preparations from HCT116 and SKOV3 cell lines. The abundance of the L1 derived peptide (corresponding to amino acids 302-311) fell below the detection limit of the instrument in the HCT116 samples indicating that SKOV3 cells possessed levels of L1 that were minimally in 10-fold greater abundance than their counterparts. Other non-L1 peptides showed little or no discernable differences in relative abundance between the two sample preparations. FIG. 2(c) shows assessment of the levels of L1 mRNA species from total RNA from the cell lines by real-time PCR analysis.

FIG. 3a-d shows apoptotic Effects of GSE and siRNA species against L1-NCAM in SKOV3 cells. FIG. 3(a) shows induction of apoptosis in a stable cell line of SKOV3 cells containing an inducible GSE against L1 as assessed by FACS analysis of active caspase-3 levels. Bars represent the percentage of cells stained positive for the caspase protein relative to background levels of isotype control stained cells. FIG. 3(b) shows analysis of L1 surface expression levels in response to doxycycline treatment in stable cell lines containing only an empty vector (top panel) or cells harboring the vector expressing the L1 GSE (bottom panel). Each panel contains the histogram profiles of doxycycline treated cells (the heaviest weighted lines) versus the untreated cells (medium weighted line). The histogram of isotype control staining is shown by the lightest weight line. FIG. 3(c) shows nduction of apoptosis in SKOV3 cells transfected with an siRNA species against L1 or an unrelated non-specific control duplex as assessed by FACS analysis of active caspase-3 levels. FIG. 4(d) shows determination of siRNA specificity in SKOV3 cells by monitoring levels of surface L1 after transfection. Histograms of L1 treated cells (heaviest weighted line) show a decrease of L1 levels relative to cells treated with the unrelated non-specific control siRNA (medium weighted line); the isotype control is the lighest weighted line in the panel.

FIG. 4a-c shows real-time PCR analysis of L1 RNA levels across a broad spectrum of normal human tissues. FIG. 4(a) show an illustration of L1 protein domains detailing the relative positions of exon 2 and exon 27. The three quantitative real time PCR (Q-PCR) primer and probe sets used in these experiments are also detailed on the diagram. FIG. 4(b) shows an assessment of the levels of L1 mRNA species from total RNA of 24 distinct normal human tissues by real-time PCR analysis using primers and probes against a region of L1 invariantly expressed in all isoforms studied to date. FIG. 4(c) shows a schematic of sequences at the junctions of exon 2 and exon 27; the forward primer spans across sequences of exon 2 and the Taqman probe spans across sequences of exon 27.

FIG. 5. shows distribution of L1 antigen as determined by immunohistochemistry analysis on a panel of normal human tissues. Deposition of DAB chromagen, specified by the brown stain, is an indication of the presence of L1 within the tissues. The tissues were counter-stained with Mayer's Hematoxylin in order to visualize the nuclei and membranes of individual cells.

FIG. 6. shows real-time PCR analysis of clinically derived diseased tissues. An independent set of Q-PCR primers and probe sets were designed to correspond against a region of the L1 gene in all identified isoforms. Real-time PCR analysis of clinically-derived human diseased tissue versus adjacent matched benign tissue. Each bar on the chart represent.

### DESCRIPTION OF THE INVENTION

The invention provides nucleic acid molecules and polypeptides identified as having a functional role in apoptosis. The invention also provides methods for using the nucleic acid molecules and polypeptides of the invention, for example, as biomarkers, therapeutics and targets for therapeutics.

In one aspect, the invention relates to isolated nucleic acid molecules identified using the genetic screen of the invention. The nucleic acid molecules may be genomic DNA, cDNA, or mRNA. In particular, the invention relates to nucleic acid molecules that correspond to L1. Another aspect of the invention relates to fragments of the nucleic acid molecules of the invention, modified nucleic acids molecules of the invention, molecules that hybridize to nucleic acid molecules of the invention and molecules that comprise the nucleic acid molecules of the invention. As used herein, the term “nucleic acid molecules of the invention” refers to all of the molecules described in this paragraph. As used herein, the term “isolated nucleic acid molecule” refers to a nucleic acid molecule that has been removed from its natural milieu (i.e., a molecule that has been subject to human manipulation) and can include DNA, RNA, or derivatives of either DNA or RNA. An isolated nucleic acid molecule can be isolated from its natural source or can be produced using recombinant DNA technology (e.g., polymerase chain reaction amplification) or chemical synthesis. Isolated nucleic acid molecules include natural nucleic acid molecules and homologs thereof, including, but not limited to, natural allelic variants and modified nucleic acid molecules in which nucleotides have been inserted, deleted, substituted, or inverted in such a manner that such modifications do not substantially interfere with the nucleic acid molecule's ability to control cell growth.

It should also be appreciated that reference to an isolated nucleic acid molecule does not necessarily reflect the extent of purity of the nucleic acid molecule. Nucleic acid molecules can be isolated and obtained in substantial purity, generally as other than an intact chromosome. Usually, the nucleic acid molecule will be obtained substantially free of other nucleic acid sequences, generally being at least about 50%, and usually at least about 90% pure. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably.

According to the invention, reference to an “isolated nucleic acid molecule” refers to a nucleic acid molecule that is the size of or smaller than a gene. Thus, an isolated nucleic acid molecule does not encompass isolated genomic DNA or an isolated chromosome. The term isolated nucleic acid molecule does not connote any specific minimum length. As used herein, the term “gene” has the meaning that is well known in the art, that is, a nucleic acid sequence that includes the translated sequences that code for a protein (“exons”) and the untranslated intervening sequences (“introns”), and any regulatory elements ordinarily necessary to translate the protein.

“Hybridization” has the meaning that is well known in the art, that is, the formation of a duplex structure by two single-stranded nucleic acids due to complementary base pairing. Hybridization can occur between exactly complementary nucleic acid strands or between nucleic acid strands that contain some regions of mismatch.

Another aspect of the invention relates to the polypeptides that are encoded by the nucleic acid molecules of the invention. Included within this aspect of the invention are fragments of the polypeptides of the invention, modified polypeptides of the invention, and molecules that comprise the polypeptides of the invention such as fusion proteins. Precursors of a polypeptide of the invention, metabolites of a polypeptide of the invention, a modified polypeptide of the invention and a fusion protein comprising all or a portion of a polypeptide of the invention are included in this aspect of the invention. As used herein, the term “polypeptide molecules of the invention” refers to all of the molecules described in this paragraph.

Another aspect of the invention relates to antibodies, antibody fragments, or other molecules that specifically recognize and bind to a polypeptide of the invention. Such molecules can be used, for example, in methods for detecting polypeptides of the invention, or in methods for treatment of cancer or other disease.

Another aspect of the invention provides molecules that modulate nucleic acid molecules or polypeptides of the invention. The modulation may be an increase or a decrease in the abundance, expression or activity of the nucleic acid molecule or polypeptide.

Another aspect of the invention relates to compositions comprising a polypeptide of the invention, a nucleic acid molecule of the invention, an inhibitor of, antibody to or modulator of a polypeptide of the invention or a nucleic acid of the invention. Such compositions may be pharmaceutical compositions in which the polypeptide, nucleic acid molecule, inhibitor, antibody or modulator is formulated for introduction into the body as a therapeutic. Pharmaceutically-acceptable carriers are well known to those with skill in the art.

Another aspect of the invention provides methods for determining the concentration, presence or activity of a polypeptide or nucleic acid of the invention. The determination may be achieved by any method known in the art. For example, the presence of a polypeptide can be determined by histological staining of tissue. Methods for determining the concentration, presence or activity of a polypeptide of the invention or a nucleic acid of the invention could be used in the diagnosis, staging, imaging or other characterization of a cancer or other disease. Such methods may be used, for example, to determine the relative distribution of a polypeptide or nucleic acid molecule among various tissues.

A further embodiment of the invention is a method for inducing apoptosis in a cell by inhibiting a target of the present invention, i.e., L1. For example, this method can be conducted in vivo by administering to an individual an inhibitory or therapeutic compound as generally discussed herein. In addition, the method can be conducted in vitro.

Another aspect of the invention relates to methods for diagnosing a cancer or other disease based on a determination of the concentration, presence or activity of a polypeptide of the invention or nucleic acid molecule of the invention. In particular, the invention relates to methods for diagnosing an ovarian, cervical or uterine cancer. A further embodiment of the present invention is a method for the diagnosis of a tumor that includes determining the level of a marker in a patient sample, wherein the marker is L1. The level of the marker can be determined by conventional methods such as expression assays to determine the level of expression of the gene, by biochemical assays to determine the level of the gene product, or by immunoassays. If appropriate, the marker can be identified as a cell surface molecule in tissue or in a bodily fluid, such as serum. For example, a patient sample, which can be immobilized, can be contacted with an antibody, or an antibody fragment, that binds specifically to the marker and determining whether the anti-marker antibody or fragment thereof has bound to the marker. In a particular immunoassay, the marker level is determined using a first monoclonal antibody that binds specifically to the marker and a second antibody that binds to the first antibody.

If the level of the marker is greater than a normal level, the level of the marker is considered to be indicative of the presence of tumor cells. A normal level can be determined in a variety of ways. For example, if a patient history is known, a baseline level of the marker can be determined and higher levels will be indicative of tumor cells. Alternatively, a normal level can be based on the level for a healthy (i.e., without tumor) individual in a given population. That is, a normal level can be based on a population having similar characteristics (e.g., age, sex, race, medical history) as the patient in question.

This method of diagnosis can be used specifically to determine the prognosis for cancer in the patient or to determine the susceptibility of the patient to a therapeutic treatment

Another aspect of the invention relates to methods for treating a cancer or other disease in a subject by providing to the subject a composition comprising a polypeptide of the invention, a nucleic acid molecule of the invention, an inhibitor of, antibody to or modulator of a polypeptide of the invention or a nucleic acid of the invention is provided to the subject. In particular, the invention relates to methods for treating ovarian, cervical or uterine cancer in a subject by providing to the subject a composition of the invention. In one embodiment, for example, the method comprises providing a composition comprising a molecule that inhibits a polypeptide of the invention. In another embodiment, the method comprises providing a nucleic acid molecule of the invention to compensate for a defective gene.

The underlying scientific basis for the aspects of the invention described above is known in the art and such aspects are enabled by differential gene expression data, as disclosed herein (Salceda et al. 2003). Other objects and advantages will become apparent to one of skill in the art from the present disclosure.

The present invention is based, in part, on the Applicants' isolation of certain GSEs from human cells that prevent cell growth, and that such nucleic acid molecules correspond to fragments of certain human cellular genes. In that regard, any cellular phenotype or protein associated with cell growth can be used to select for such nucleic acid molecules. GSEs having the ability to control cell growth can be functional in the sense orientation (and encode a peptide thereby), and can be functional in the antisense orientation (and encode antisense RNAs thereby). These GSEs are believed to down-regulate the corresponding cellular gene from which they were derived by different mechanisms. Such a corresponding cellular gene is referred to herein as a “target gene” and its product is referred to as a “target product.” As used herein, the term “target” alone refers collectively to a target gene and its corresponding target product. Sense-oriented GSEs exert their effects as transdominant mutants or RNA decoys. Transdominant mutants are expressed proteins or peptides that competitively inhibit the normal function of a wild-type protein in a dominant fashion. RNA decoys are protein binding sites that titrate out these wild-type proteins. Anti-sense oriented GSEs exert their effects as antisense RNA molecules, i.e., nucleic acid molecules complementary to the mRNA of the target gene. These nucleic acid molecules bind to mRNA and block the translation of the mRNA. In addition, some antisense nucleic acid molecules can act directly at the DNA level to inhibit transcription. A specific target gene is the gene for L1. The products of the target gene is a target product of the present invention. Methods of the present invention for identifying therapeutic compounds by identifying an inhibitor of a target in the human host cell include identifying an inhibitor of L1.

In one embodiment of the invention, the down-regulation of the concentration or activity of a target gene or product by an inhibitor (including a GSE) depletes a cellular component required for protecting cells from apoptosis resulting in control of cell growth. In another embodiment of the invention, the down-regulation of the concentration or activity of one target gene or product by an inhibitor (including a GSE) depletes a cellular component that interacts with another human cellular gene or gene product required for protecting cells from apoptosis resulting in control of cell growth. In one embodiment of the invention, the two human cellular genes are members of the same biological pathway and one human cellular gene or gene product regulates the expression or activity of the other human cellular gene or gene product. In another embodiment of the invention, the two human cellular genes are members of the same biological pathway and the substrate of a polypeptide encoded by one human cellular gene is a product of a biochemical reaction mediated by the polypeptide encoded by the other human cellular gene. In still another embodiment of the invention, the two human cellular genes are members of the same biological pathway and the product of a polypeptide encoded by one human cellular gene is a substrate of a biochemical reaction mediated by the polypeptide encoded by the other human cellular gene. In another embodiment, the two human cellular genes encode polypeptides that are isozymes of each other. In a embodiment, at least one of the human cellular genes encodes an enzyme.

It will be understood that this invention is not limited to the particular methodology, protocols, cell lines, animal species or genera, constructs, or reagents described herein, as such may vary. It is also to be understood that the terminology used herein is for the purpose of describing particular embodiments only, and is not intended to limit the scope of the invention that will be limited only by the appended claims. All technical and scientific terms used herein have the same meaning as commonly understood to one of ordinary skill in the art to which this invention belongs unless clearly indicated otherwise.

Target genes or proteins identified using GSEs can be further evaluated using a variety of methods to validate their involvement in cell growth, suppression of apoptosis and tumor formation. Such methods include methods that disrupt or “knock out” the expression of a target gene in a cell capable of apoptosis. Knock-out methods include somatic cell knock-outs and inhibitory RNA molecules including anti-sense oligonucleotides, siRNA molecules, RNAi molecules and RNA decoys. Target genes or proteins can also be evaluated by methods that include nucleic acid-based experiments such as Northern Blots, Real Time polymerase chain reaction or high density microarrays. Further evaluation can also be achieved using human/mouse xenograft models. For example, human tumor cells can be transfected with a GSE such that the GSE is expressed. Tumor cells include HCT116 and MDA-MB-231. The transfected cells can then be implanted into mice, including nude mice. The growth of the tumor cells in the mouse can then be measured.

Once one or more members of a biological pathway are identified as required for cell growth, the present invention can include identifying additional members of a biological pathway that are also required for cell growth. Such subsequent identification is within the skill of one in the art. GSEs, and therefore targets of the present invention, are identified by selecting cells that exhibit certain hallmarks of apoptosis upon expression of the GSEs. Isolated GSEs are further prioritized based on their specificity for a neoplastic transformation state, such as their activity in transformed and non-transformed cells, and based on the p53 pathway status in cells expressing the GSEs. For example, GSEs can be prioritized by determining if the GSEs have activity in an L1-dependent and/or independent manner. GSEs specific for the neoplastic transformation state are useful for identifying targets for anti-cancer drugs.

Once a human cellular gene has been identified as a target for supporting cell growth, an assay can be used for screening and selecting a chemical compound or a biological compound having activity as an anti-tumor therapeutic based on the ability to down-regulate expression of the gene or inhibit activity of its gene product. Reference herein to inhibiting a target, refers to both inhibiting expression of a target gene and inhibiting the activity of its corresponding expression product. Such a compound is referred to herein as therapeutic compound. For example, a cell line that naturally expresses the gene of interest or has been transfected with the gene is incubated with various compounds. A reduction of the expression of the gene of interest or an inhibition of the activities of its encoded product may be used as to identify a therapeutic compound. Therapeutic compounds identified in this manner can then be re-tested in other assays to confirm their activities against apoptosis.

In one embodiment of the invention, inhibitors of cell growth are identified by exposing a mammalian cell to a test compound; measuring the expression of a human cellular gene or an activity of the polypeptide encoded by the human cellular gene in the mammalian cell; and selecting a compound that down-regulates the expression of the human cellular gene or interferes with the activities of its encoded product. One mammalian cell to use in an assay is a mammalian cell that either naturally expresses the human cellular gene or has been transformed with a recombinant form of the human cellular gene. Methods to determine expression levels of a gene are well known in the art.

In one embodiment, the expression of the human cellular gene is measured by the polymerase chain reaction. In another embodiment, the expression of the human cellular gene is measured using an antibody that specifically recognizes the polypeptide encoded by the human cellular gene and is analyzed using methods such as immunoprecipitation, ELISAs, fluorescence activated cell sorting (FACS) and immunofluorescence microscopy. In another embodiment, the expression of the human cellular gene is measured using polyacrylamide gel analysis, chromatography or spectroscopy. In still another embodiment, the activity of the polypeptide encoded by the human cellular gene is measured by measuring the amount of product generated in a biochemical reaction mediated by the polypeptide encoded by the human cellular gene. In still another embodiment, the activity of the polypeptide encoded by the human cellular gene is measured by measuring the amount of substrate generated in a biochemical reaction mediated by the polypeptide encoded by the target gene. In another embodiment of the invention, therapeutic compounds are selected by determining the three-dimensional structure of a human cellular gene product; and determining the three-dimensional structure of a therapeutic compound by rational drug design. In some cases, the structure of the therapeutic compound is determined using computer software capable of modeling the interaction of a therapeutic compound with the target gene. One of skill in the art can select the appropriate three-dimensional structure, therapeutic compound, and analytical software based on the identity of the target gene.

In still another embodiment of the invention, inhibitors of cell growth are identified by exposing a polypeptide encoded by a target gene to a test compound; measuring the binding of the test compound to the polypeptide; and selecting a compound that binds to the polypeptide at a desired concentration, affinity, or avidity. In one embodiment, the assay is performed under conditions conducive to promoting the interaction or binding of the compound to the polypeptide. One of skill in the art can determine such conditions based on the polypeptide and the compound being used in the assay.

In still another embodiment of the invention, a therapeutic compound is identified by exposing an enzyme encoded by a target gene to a test compound; measuring the activity of the enzyme encoded by the target gene in the presence and absence of the compound; and selecting a compound that down-regulates or inhibits the activity of the enzyme encoded by the target gene. Methods to measure enzymatic activity are well known to those skilled in the art and are selected based on the identity of the enzyme being tested. For example, if the enzyme is a kinase, phosphorylation assays can be used.

In addition to methods for identifying and producing a biological compound that inhibits cell growth, the present invention includes methods known in the art that down-regulate expression or function of a target gene. For example, antisense RNA and DNA molecules may be used to directly block translation of mRNA encoded by these cellular genes by binding to targeted mRNA and preventing protein translation. Polydeoxyribonucleotides can form sequence-specific triple helices by hydrogen bonding to specific complementary sequences in duplexed DNA to effect specific down-regulation of target gene expression. Formation of specific triple helices may selectively inhibit the replication or expression of a target gene by prohibiting the specific binding of functional trans-acting factors.

Ribozymes are enzymatic RNA molecules capable of catalyzing the specific cleavage of RNA. Ribozyme action involves sequence specific hybridization of the ribozyme molecule to complementary target RNA, followed by endonucleolytic cleavage. Within the scope of the invention are ribozyme embodiments including engineered hammerhead motif ribozyme molecules that specifically and efficiently catalyze endonucleolytic cleavage of cellular RNA sequences. Antisense RNA molecules showing high-affinity binding to target sequences can also be used as ribozymes by addition of enzymatically active sequences known to those skilled in the art.

Polynucleotides to be used in triplex helix formation should be single-stranded and composed of deoxynucleotides. The base composition of these polynucleotides must be designed to promote triple helix formation via Hoogsteen base pairing rules, which generally require sizeable stretches of either purines or pyrimidines to be present on one strand of a duplex. Polynucleotide sequences may be pyrimidine-based, which will result in TAT and CGC triplets across the three associated strands of the resulting triple helix. The pyrimidine-rich polynucleotides provide base complementarity to a purine-rich region of a single strand of the duplex in a parallel orientation to that strand. In addition, polynucleotides may be chosen that are purine-rich, for example, containing a stretch of G residues. These polynucleotides will form a triple helix with a DNA duplex that is rich in GC pairs, in which the majority of the purine residues are located on a single strand of the targeted duplex, resulting in GGC triplets across the three strands in the triplex.

Alternatively, sequences that can be targeted for triple helix formation can be increased by creating a so-called “switchback” polynucleotide. Switchback polynucleotides are synthesized in an alternating 5′-3′,3′-5′ manner, so that they base pair with first one strand of a duplex and then the other, eliminating the necessity for a sizeable stretch of either purines or pyrimidines to be present on one strand of a duplex.

Both antisense RNA and DNA molecules, and ribozymes of the invention may be prepared by any method known in the art. These include techniques for chemically synthesizing polynucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into host cells.

Various modifications to the nucleic acid molecules may be introduced as a means of increasing intracellular stability and half-life. Possible modifications include, but are not limited to, the addition of flanking sequences of ribonucleotides or deoxyribonucleotides to the 5′ or 3′ ends of the molecule or the use of phosphorothioate or 2′ O-methyl rather than phosphodiesterase linkages within the oligodeoxyribonucleotide backbone.

Methods used to identify therapeutic compounds may be customized for each target gene or product. If the target product is an enzyme, then the enzyme will be expressed in cell culture and purified. The enzyme will then be screened in vitro against therapeutic compounds to look for inhibition of that enzymatic activity. If the target is a non-catalytic protein, then it will also be expressed and purified. Therapeutic compounds will then be tested for their ability to prevent, for example, the binding of a site-specific antibody or a target-specific ligand to the target product.

In one embodiment, therapeutic compounds that bind to target products are identified, then those compounds can be further tested in biological assays that test for characteristics such as apoptosis, p53 status, tumor cell growth and any other customary measure of anti-cancer activity.

In one embodiment of the invention, a therapeutic compound is not toxic to a human host cell. In another embodiment, the therapeutic is cytostatic or cytotoxic.

In a genetic screen, a functional role was identified for L1 in regulating cell growth and apoptosis in cancer cell lines. Down regulation of L1 expression levels by genetic suppressor elements and small interfering ribonucleic acid (siRNA) caused the induction of apoptosis in cancer cells derived from non-neuronal tumors. A thorough analysis of L1 mRNA and protein distribution across a large panel of normal human tissues revealed a diverse distribution, including the presence of several L1 isoforms that were previously reported to be restricted to neuronal or diseased tissue. Furthermore, analyses of a wide variety of cancer cell lines as well as patient tissue samples indicate an abundant expression pattern of L1 within tumors of the ovary, cervix and uterus. These findings indicate an important role for L1 in cancer, and make L1 an important target for diagnosis of cancer, and for the development of therapeutics.

Role of L1 in Apoptosis

A genetic screen was used to identify genes implicated in the regulation of cancer cell growth and apoptosis. A retroviral GSE expression library was constructed from cancer cells and used to transduce the colon carcinoma cell line HCT116. GSEs that induce caspase-3, an early marker for cells undergoing apoptosis were selected and subsequently identified by DNA sequencing. A detailed description of the genetic screen is provided, e.g., in United States Patent Application Publication No. 2004/0170989 A1 and United States Provisional Patent Application No., 60/539,167. L1 was one of the genes identified from the genetic screen.

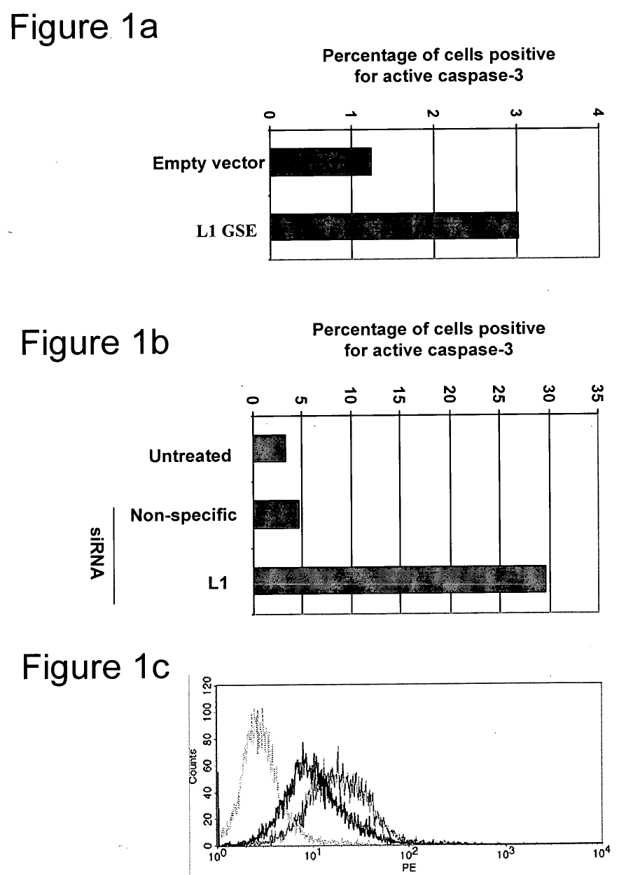

To provide further confirmation of the functional role of L1 in apoptosis, HCT116 cells were engineered to express an L1 GSE under the control of a doxycycline-inducible expression vector system. The cells were induced for 48 hours by doxycycline and apoptosis was measured by monitoring the levels of active caspase-3. Cells expressing the L1 GSE demonstrated modest, but reproducible, increases in apoptosis as compared to cells expressing an empty vector. FIG. 1a.

In addition, the effects of an siRNA species derived from L1 were tested in the HCT116 cell line. The cells were transfected with either an siRNA species directed against L1 or a control siRNA duplex that does not correspond to any known human sequence. Following a 72-hour incubation period after transfection, cells were harvested and assayed for the relative levels of active caspase-3. As shown in FIG. 1b, greater than 29% of the cells transfected with the siRNA duplex specific for L1 stained positive for the active caspase-3 species, compared to 3.0% and 4.4% of untreated cells or cells transfected with the non-specific siRNA control. To confirm the specificity of the siRNA, levels of L1 surface expression were monitored following treatment with the siRNA. Expression of surface L1 was reduced in cells transfected with the L1 siRNA species but remained unchanged in response to the non-specific siRNA. FIG. 1c.

It has been reported in the literature that a fusion protein comprised of the L1 extracellular domain and the Fc region of immunoglobulin conferred upon cerebellar and hippocampal neurons the ability to resist apoptosis when cultured under serum-free conditions (Chen et al., 1999). While the results of that study suggest an abundance of L1 confers a protective role against apoptosis, the findings of the present invention demonstrate that down regulation of the protein can stimulate apoptosis.

It has also been reported in the literature that GSEs against L1 were isolated from a genetic screen set up to identify elements which could inhibit the proliferation of MDA-MB-231 cells (Primiano et al., 2003). The results of that study may be explained by the findings of the present invention that GSEs against L1 induce apoptosis—by inducing apoptsis, GSEs against L1 affect the replicative potential of the population of cells as a whole.

It has been previously shown that L1 mutations which result in the down regulation of the surface protein levels lead to the severe pathogenic phenotypes often associated with syndromes like CRASH (Runker et al., 2003). Furthermore, the severity of the disease directly correlates with the relative levels of L1 cell surface expression (Weller et al., 2001). The results of the present invention suggest that some of the phenotypic effects which are associated with CRASH or MASA syndrome may be linked to apoptosis of the cells in which L1 is aberrantly expressed.

L1 Expression in Ovarian and Cervical Cancer Cell Lines

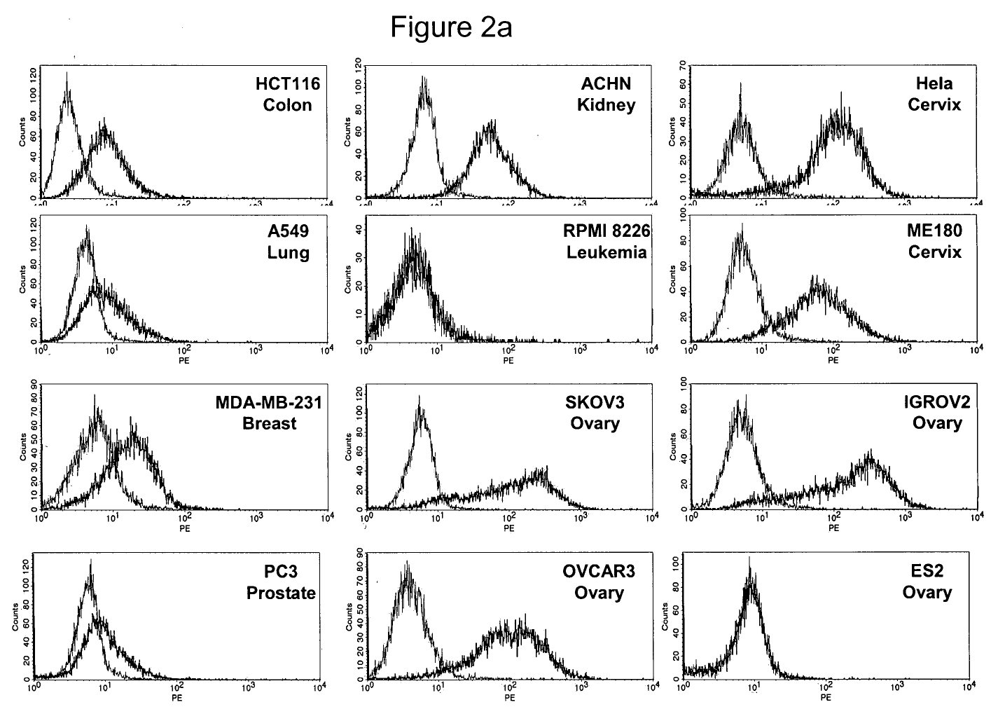

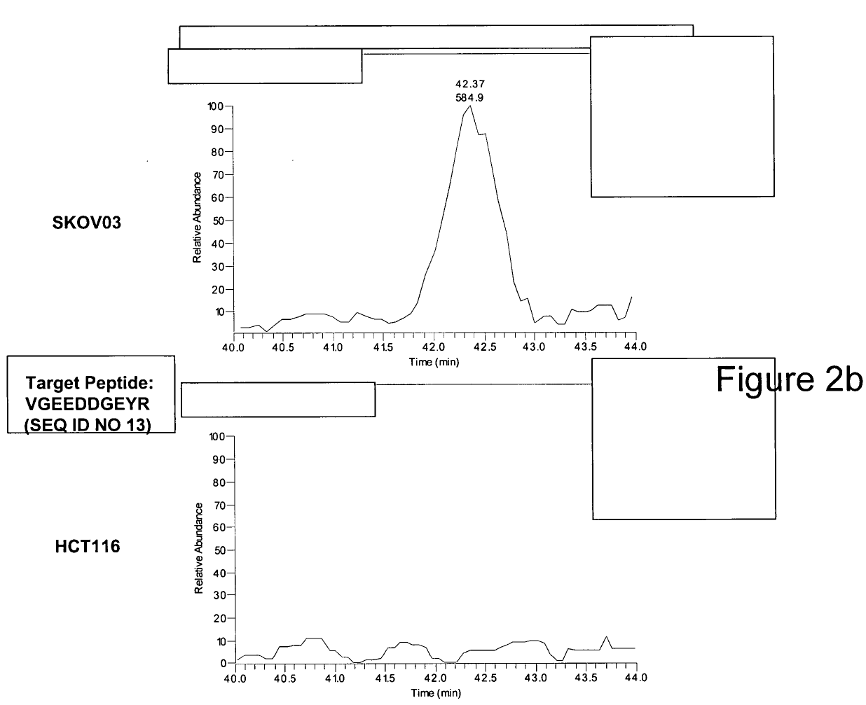

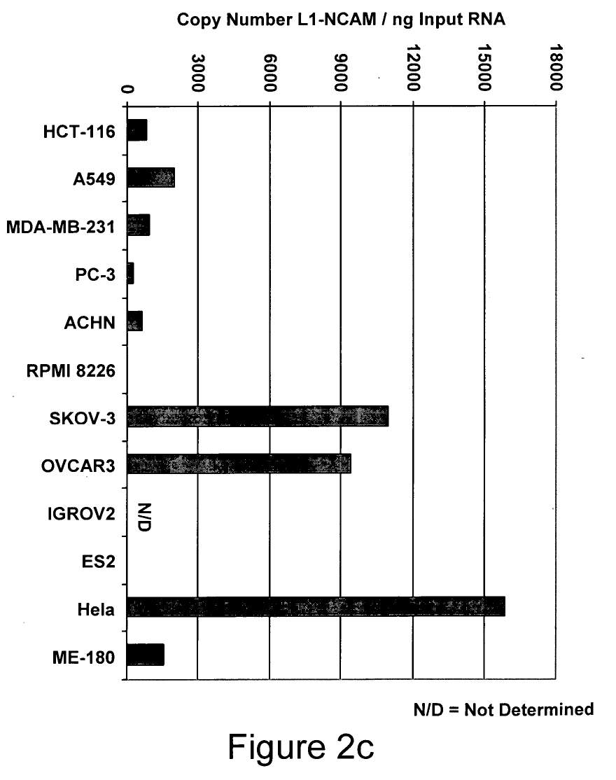

The distribution of L1 was investigated across a wide variety of transformed human cell lines and human tissues by analyzing the relative expression patterns of its protein and mRNA levels (“L1 expression”). As shown in FIG. 2a, high levels of L1 expression were detected in several ovarian-derived cells including SKOV3, OVCAR3 and IGROV2; though also originating from ovarian tissues, ES2 cells appeared to be devoid of L1 expression. A high level of expression was also noted in the cervix-derived HeLa and ME180 cell lines as well as the renal-based ACHN cell line. Significantly lower levels of L1 were detected in colon, lung, breast and prostate cell lines; little or no expression was noted in the leukemia cell line RPMI-8226.

Independent confirmation of L1 expression levels in several cell lines was performed by liquid chromatograph-mass spectrometry analysis (LC-MS). Fractions of enriched plasma membranes were isolated by subcellular fractionation techniques and subjected to LC-MS. Consistent with the FACS analysis, a peptide derived from L1 was detected in SKOV3 preparations at levels greater than ten-fold excess of an analogous extract from the HCT116 cell line. FIG. 2b.

To further characterize the expression of L1, the relative abundance of its mRNA levels in the cells lines was measured using quantitative real-time PCR analysis (Q-PCR). Consistent with the protein expression data, a high level of the transcript was detected in the ovarian cell lines SKOV3 and OVCAR3 and the cervix-based HeLa cell line. FIG. 2c. Significantly reduced levels of L1 mRNA were detected in the remainder of the cell lines.

Recent studies analyzed the expression levels of L1 in diseased tissues. Some of the strongest expression levels of L1 have been measured in metastatic tumors and other diseased tissues such as malignant melanoma (Fogel et al., 2003a). In the present invention, an abundance of L1 protein expression was noted in cell lines derived from cancerous lesions of ovarian and cervical tissues. FIG. 2. For example, the levels of surface L1 protein are markedly close to the levels of ErbB-2, a tyrosine kinase receptor implicated with a role in several cancers (Scholl et al., 2001), in the ovarian cancer cell line SKOV3. Because of their high levels of ErbB-2, SKOV3 cells are often used as a model for the development of anti-ErbB-2 therapeutic monoclonal antibodies. The findings of the present invention indicate that the SKOV3 model can be also used for the development of anti-L1 monoclonal antibody-based cancer therapeutics.

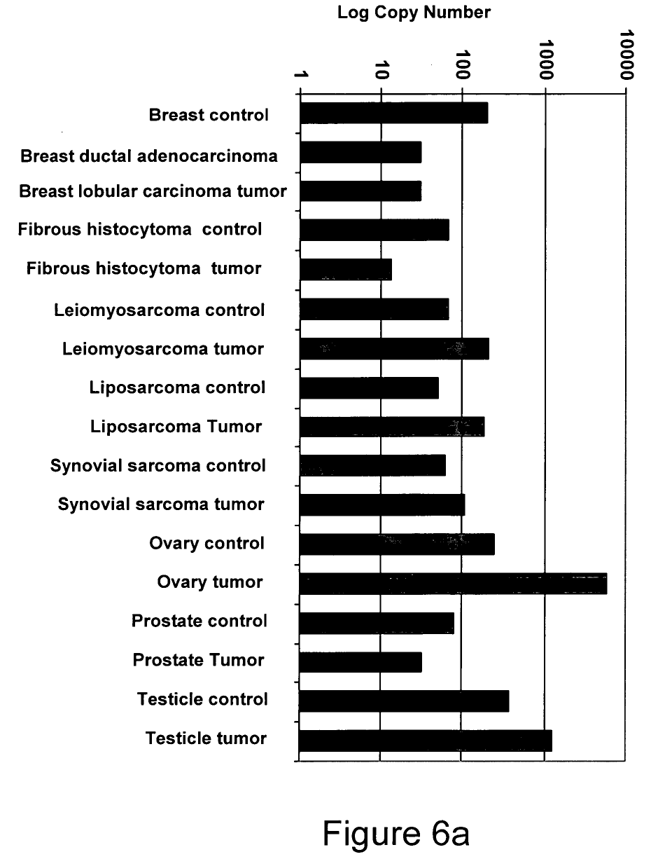

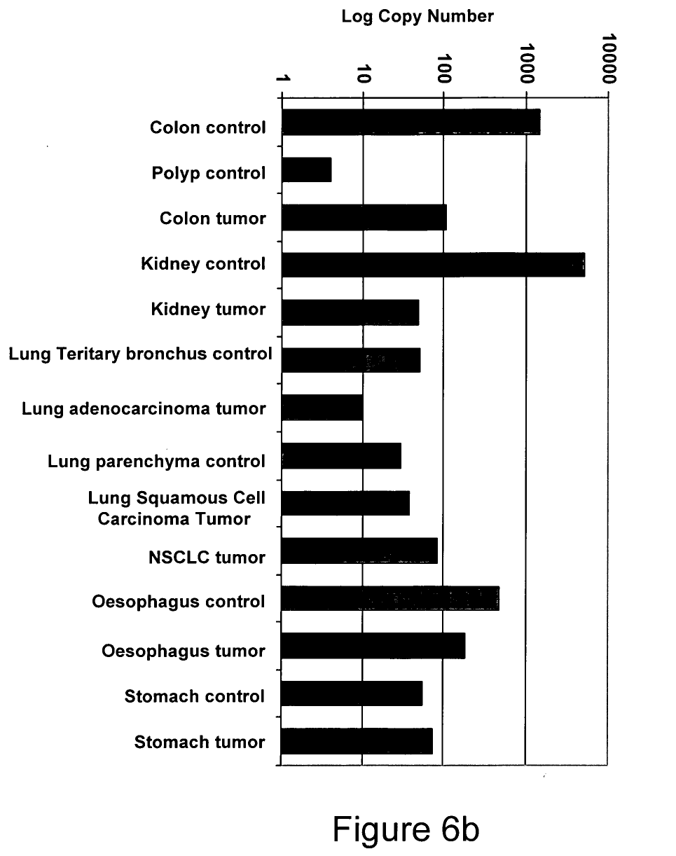

Furthermore, when tested in patient material (FIG. 6), the aberrant expression patterns of L1 in diseased tissue indicates a role for the L1 in ovarian, uterine and cervical cancers. These sets of expression data are entirely consistent with a recently published report which describes a strong correlation between the over-abundance of L1 on the surface of uterine and cervical tumors and a poor prognosis of recovery (Fogel et al., 2003b).

Thus, when considering the expression and functional data together, L1 appears to be an attractive target for the development of therapeutic monoclonal antibodies against ovarian and cervical cancer. It has been well documented that treatment of neuronal cell lines with polyclonal or monoclonal antibodies can inhibit neurite outgrowth (Kristiansen et al., 1999; Hall, 2000; Yip, 2001). Additionally, Primiano et al. demonstrated that addition of monoclonal antibodies to cell culture of non-neuronal cell lines, including the HeLa cells (utilized as well in our current study) was sufficient to inhibit cellular proliferation (Primiano et al., 2003).

A mouse-human chimeric antibody against the L1 protein, designated chCE7, has been developed and tested extensively in several pre-clinical models as a radioimmunoconjugate variant that is directed as a therapeutic against neuroblastoma (Amstutz et al., 1993; Novak-Hofer et al., 1997). However, the antibody exhibits a limited potential for use in therapeutic applications due to a lack of sustained potency. In an effort to increase duration of the potency, the chE7 Fc region was glycosylated to elicit enhanced ADCC response (Umanal et al., 1999). It has yet to be determined whether or not this specific reagent, modified or otherwise, has any utility as a therapeutic against tumors derived from ovarian or cervical tissues.

Role of L1 in Ovarian and Cervical Cell Lines

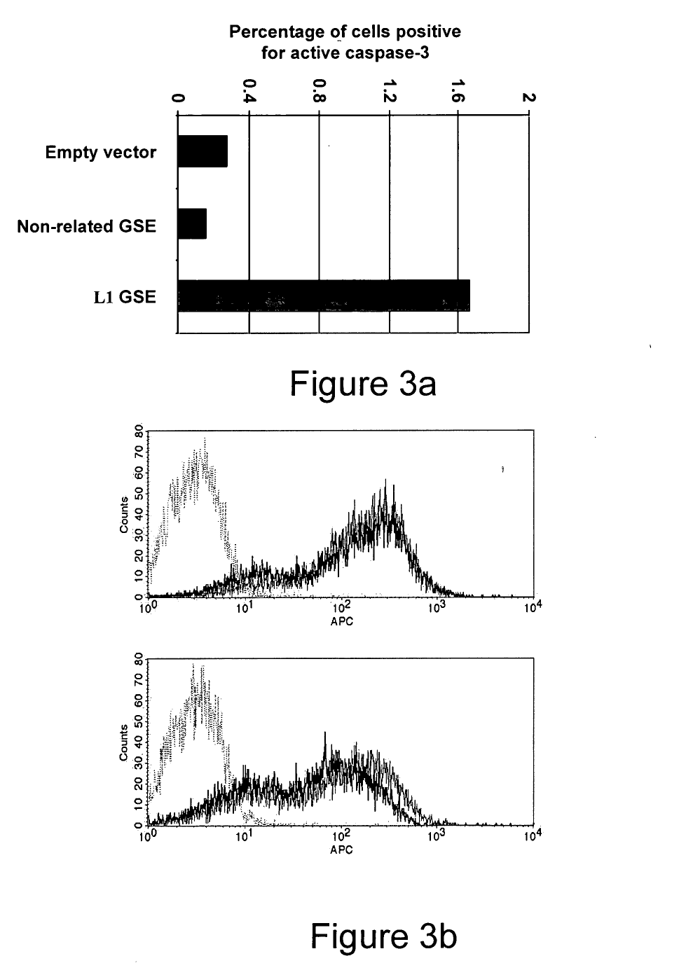

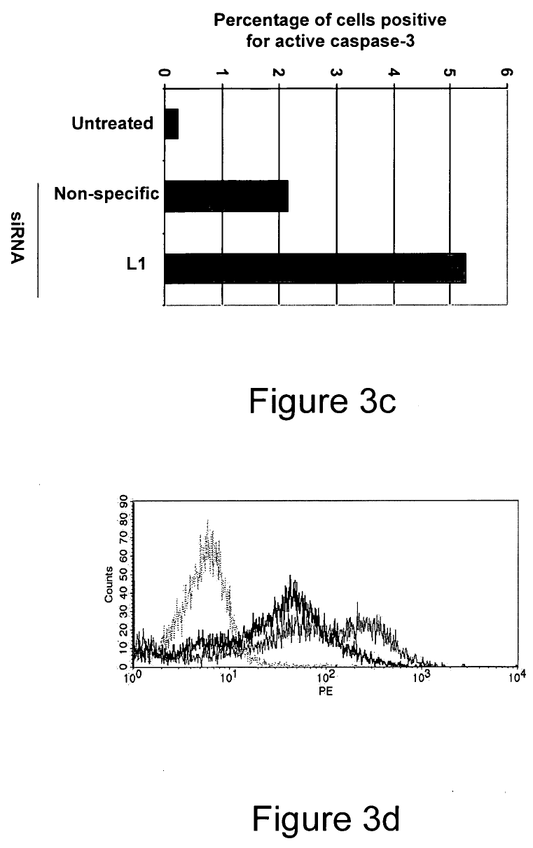

To further explore the role of L1 in cells derived from ovarian and cervical cell lines, the effects of an L1 GSE and an L1 siRNA species on apoptosis was studied in a representative cell line. SKOV3 cells were engineered to express an L1 GSE under the control of the doxycycline inducible expression vector system. Expression of the L1 GSE was induced for 72 hours by the addition of doxycycline and apoptosis was measured by monitoring the levels of active caspase-3. While the overall percentage of caspase-3 positive cells was low, cells expressing L1 GSE showed a greater than six-fold increase in apoptosis over cells expressing the empty vector control. FIG. 3a. Correspondingly, expression of surface L1 was modestly decreased in cells expressing a doxycycline inducible GSE. FIG. 3b (lower panel). Cells expressing an empty vector showed no decrease in L1 expression in response to the doxycycline treatment. FIG. 3b (upper panel).

In a comparable set of studies, SKOV3 cells were transfected with either an siRNA species directed against L1 or the non-specific control siRNA duplex. As shown in FIG. 3c, 5.3% of the cell population transfected with the L1 siRNA species stained positive for active caspase-3 as compared to 2.1% of cells transfected with the non-specific control siRNA. The specificity and efficacy of the L1 siRNA was demonstrated by its ability to reduce surface levels of the L1 protein as compared to the non-specific control. FIG. 3d. When evaluated against the results obtained with the HCT116 cell line, L1 GSE or L1 siRNA species showed a reproducible (albeit modest) ability to elicit apoptosis or decrease the level of surface protein in SKOV3 cells. The decrease in efficiency may be attributed, in part, to the vastly higher levels of L1 expression in these cells as compared to their HCT116 counterparts.

Distribution of L1 RNA in Normal Tissues

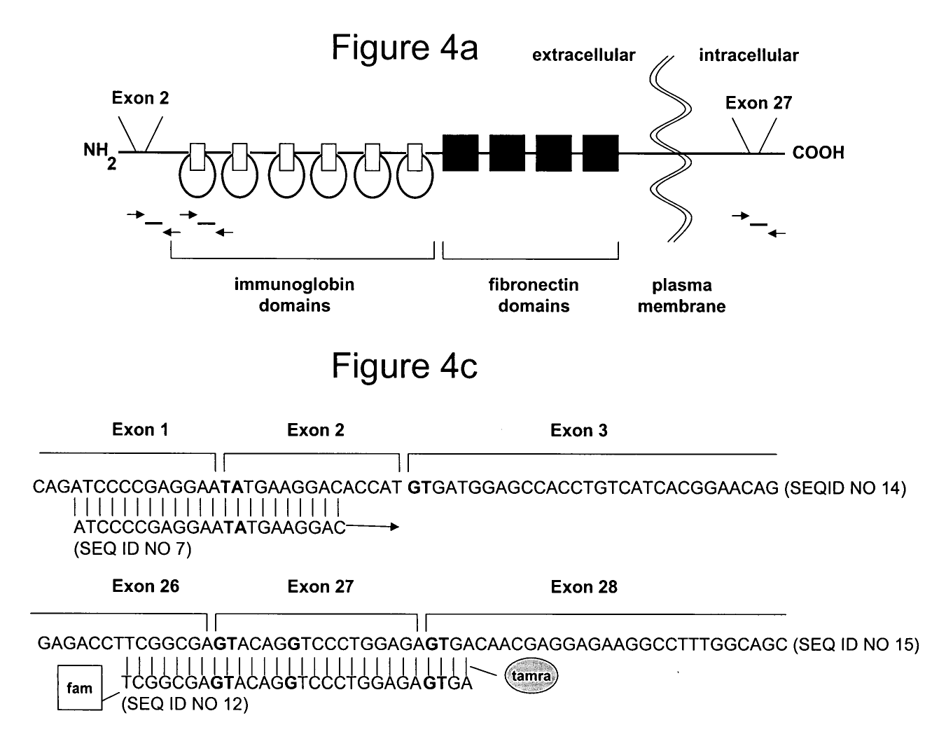

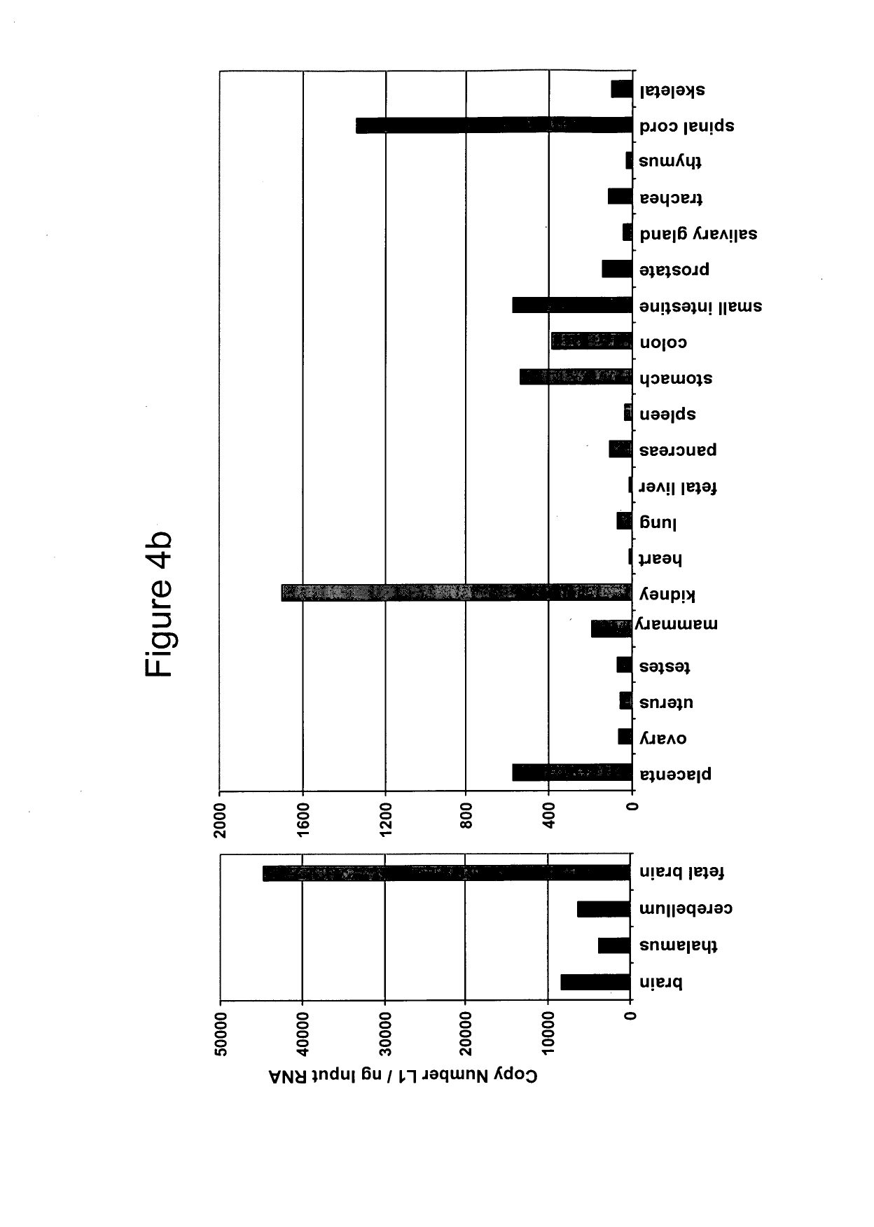

The relative distribution of L1 mRNA levels was determined in a diverse representation of normal human tissues. RNA isolated from 26 distinct tissues was interrogated by Q-PCR with a primer and probe set against a region of L1 thought to be invariably expressed across all isoforms. FIG. 4a. Relatively large concentrations of L1 mRNA were found in neuronal tissues. FIG. 4b (left panel). Consistent with a role for L1 in axonal guidance, the highest levels of corresponding RNA were found in fetal brain tissue. Significantly lower levels of L1 mRNA were found in spinal tissues. FIG. 4b (right panel). Tissues outside of the central nervous system contain L1 mRNA levels that were markedly decreased. FIG. 4b (right panel). Of the non-neuronal tissues, the highest levels of mRNA were detected in kidney; whereas, lower levels were found in tissues from the stomach, colon and the small intestine. Significantly, the normal ovarian and uterine tissues exhibit a comparatively low abundance of the L1 transcript.

While the physiological role of L1 in neuronal developmental processes is well established, the tissue distribution of L1 indicates that the protein likely plays a global role outside of neuronal tissues.

Distribution of L1 RNA Common Splice Variants in Normal Tissues

Independent sets of Q-PCR primers and probes were designed to specifically detect the presence of either exon 2 or exon 27. The real-time PCR probe or primer sequence was designed to span a portion of the sequences contained within each exon. FIG. 4c. Thus, an exon deletion was indicated by the lack of the appropriate fluorescence signal from the probe.

Standard curves were used to calibrate the signal and to normalize the data for primer binding efficiency. Thus, it was possible to directly compare the levels of transcripts obtained with the various primers. The comparison of L1 mRNA levels detected with primers and probes against a region of invariantly expressed in all L1 mRNA species versus those specific for exon 2 and exon 27 is shown in Table 1. Comparable levels of RNA were detected in the various brain tissues when using primer sets against the invariant region or against exon 2 indicating that all of the L1 transcripts detected likely contain exon 2. Similarly, comparable levels of L1 RNA were detected in four of the five brain tissues when comparing data sets obtained using a primer set against an invariant region of L1 and a primer set positioned across exon 27. By contrast, only a small fraction of the RNA purified from the thalamus possessed exon 27 indicating that the presence of this exon is not ubiquitous across all isoforms of neuronal L1. Since spinal cord tissues are comprised in part by axons, it was not unexpected that a significant portion of L1 transcripts from this sample harbored exon 2 and exon 27.

Because expression of exon 2 and 27 was thought to be limited to neuronal tissues, it was surprising that similar analyses of non-neuronal tissues demonstrated the presence of these exons in a subset of the samples. A large percentage of L1 transcript isoforms isolated from the colon and small intestine also possess exon 2 and exon 27. Not all tissues exhibit similar expression patterns of these isoforms—while stomach, kidney and placental tissues yielded modest levels of L1 transcripts, the L1 RNA species were generally devoid of exon 2 and exon 27.

The present invention demonstrates that several of the non-neuronal species contain exon 2 and exon 27, previously thought to be restricted to isoforms found within neuronal tissues or tumor tissues. For instance, Altevogt and Fogel have suggested that the detection of exon 27 in ovarian tumors may serve as a useful diagnostic marker for ovarian cancer (Altevogt, 2002). Although the levels of exon 2 or exon 27 were not directly measured in diseased tissue, the present invention clearly demonstrates the presence of these moieties in L1 mRNAs within a number of normal non-neuronal tissues. Table 1.

Immunohistochemical Analysis of L1 Protein Expression in Normal Tissues

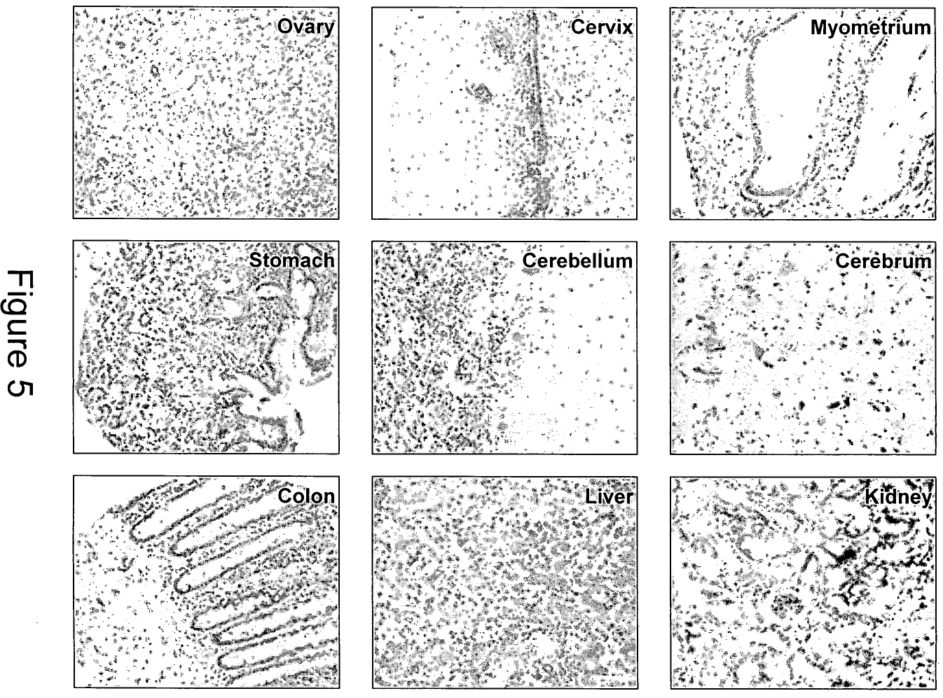

Immunohistochemical analysis was performed on an array of 24 normal tissues using an L1 monoclonal antibody. Consistent with the RNA analysis, L1 protein expression was not readily detected in normal ovary and cervical tissues, though the myometrium revealed areas of light L1 expression. FIG. 5. However, in some cases, L1 protein could not be readily detected in tissues with high RNA levels including cerebellum and other neuronal tissues. It is possible that the composition of these tissues did not allow for efficient retrieval of the antigen through the recovery techniques used. The histological samples derived from the liver, colon and kidney exhibited the highest levels of L1 antigen. The data from the latter two tissues corresponds with the relative levels of L1 mRNA detected in similar samples.

Distribution of L1 RNA in Normal and Tumor Patient Tissues

Quantitative real time PCR analysis was conducted on a number of tumors and corresponding matched normal tissues. As shown in FIG. 6a, ovarian tumors harbored a greater than 23-fold increase in L1 mRNA quantities than their normal ovarian tissue counterparts. Elevated L1 mRNA levels were detected in testicular tumors (4-fold increase) in comparison to their matched normal tissues. In other tissues such as kidney and colon, significantly greater amounts of transcript were detected in the normal tissue than their diseased counterparts. FIG. 6b.

To assess the presence of L1 protein on primary human tumors, immunohistochemical staining experiments were performed on a wide variety of ovarian, cervical and uterine tissue arrays containing both normal and diseased specimen from each of the organs. Analysis of the uterine tissues showed a segmented staining pattern in adenocarcinomas that was localized to cells adjacent to the stromal tissues. Normal uterine tissues (top panel) showed a much lighter staining pattern within a single layer of cells adjacent to the stromal tissues. Several of the adenocarcinomas isolated from the ovary also had similar segmented staining patterns (middle panel), but there were also many instances of diffuse chromagen distribution across the diseased tissues (lowest panel). In general, diseased tissues from cervical tissues that possessed significantly high levels of L1, stained heavily, but rather diffusely, for the L1 protein.

It should be noted that the foregoing description is only illustrative of the invention. Various alternatives and modifications can be devised by those skilled in the art without departing from the invention. Accordingly, the invention is intended to embrace all such alternatives, modifications and variances which fall within the scope of the disclosed invention. The Examples, which follow, are illustrative of specific embodiments of the invention, and various uses thereof. They are set forth for explanatory purposes only, and are not to be taken as limiting the invention.

### EXAMPLES

Cell lines and cell culture. All cell lines used in this study, HCT116, SKOV3, PC3, A549, MDAMB231, RPM18226, ME180, Hela, IGROV2, OVCAR3, ES2 and ACHN were obtained through the American Type Culture Collection and were maintained in media according to the directions provided with each cell line.

Stable Cell Lines. Stable cell lines with tetracycline-inducible expression of an L1 GSE were generated by transfection of an expression vector carrying a bicistronic construct encoding the renilla green fluorescent protein encephalomyocarditis virus-internal ribosomyl entry site-L1 (GFP-ECMV-IRES-L1) GSE cassette into HCT116 or SKOV3 clonal cell lines stably expressing tetracycline repressor (TetR) protein. TetR protein was expressed from pcDNA6/TR vector (T-REx™ System, Invitrogen). 48 hours after transfection cells were replated into selection medium containing 200 mg/ml hygromycin and 10 mg/ml blasticidin S. After 10-14 days of selection, selected colonies were pooled, expanded and hrGFP-IRES-GSE expression was induced by doxycycline treatment (1 mg/ml, 15 hours). Induced cells were sorted (FACSVantage, BD Bioscience) to isolate GFP-positive populations (SPs) which were further expanded in the absence of doxycycline. Inducible expression of the hrGFP-IRES-GSE cassette was confirmed by FACS and real-time PCR analysis. After a 10-14 day selection period, individual colonies were picked and expanded to produce clonal cell lines with inducible GSE expression. Apoptosis mediated by expression of the L1 GSE in these stable cell lines was measured by a FACS assay measuring the relative quantity of active caspase-3. GSE expression was induced by addition of 1 mg/ml doxycycline at 24 hours after plating. Following 48 or 72 hours of doxycycline treatment cells were harvested, the floating and attached cells combined, fixed in Cytofix/Cytoperm solution (BD Pharmingen) and stained with phycoerythrin (PE)-conjugated antibody against active caspase-3 (BD Pharmingen). Data were collected by on a FACSCalibur system (Becton Dickinson) and analyzed using CellQuest (Becton Dickinson) software.

FACS Staining. The monoclonal antibody clone UJ127.11(LabVision), with reactivity against the extracellular domain, was used to detect surface L1 protein. Zenon Antibody Labeling Kits (Molecular Probes) were used to fluorescently label the primary antibody with phycoerythrin (PE) or allophycocyanin (APC) for detection by FACS analysis. Non-specific staining was assessed by utilization of an APC- or PE-conjugated mouse IgG1 isotype control antibody. Data collection and analysis were performed using BD CellQuest Pro software on a FACSCalibur System (Becton Dickinson).

RNAi. The L1 siRNA complexes used in these studies were designed to according to the set of guidelines established by the Tuschl laboratory (Elbashir et al., 2001b; Elbashir et al., 2001c). Single strands of complementary 21-nucleotide RNA with an overhang of 2 deoxynucleotides on the 3′ termini were synthesized (Proligo). Sequences used include UGGUACAGUCUGGGCAAGGTT (SEQ ID NO:17); CCUUGCCCAGACUGUACCATT (SEQ ID NO:18); CAGCAACUUUGCUCAGAGGTT (SEQ ID NO:19); CCUCUGAGCAAAGUUGCUGTT (SEQ ID NO:20); GAAAGGUUCCAGGGUGACCTT (SEQ ID NO:21); and GGUCACCCUGGAACCUUUCTT (SEQ ID NO:22). One of two different RNAi duplexes was used for each of the L1 studies, identified by the sequence to the sense strand: 5′-TGGTACAGTCTGGGCAAGGdTdT-3′ (SEQ ID NO:1) and 5′-CAGCAACTTTGCUCAGAGGdTdT-3′ (SEQ ID NO:2). For each duplex, strands were independently resuspended in annealing buffer (10 mM Tris-HCl, pH 8.3; 0.2 mM MgCl2; 50 mM KCl) at a final concentration of 20 μM. To generate annealed siRNA duplexes, equivalent volumes of each RNA strand solution were combined and heated to 90° C. for 1 minute in a heat block which was then turned off and allowed to cool to room temperature. For transfection experiments, HCT116 and SKOV3 cells were plated the day before transfection with antibiotic-free media into either a 6-well plate format at a density of 5×10⁴ or 1.5×10⁴ cells per well respectively. 5 μL of each 20 μM siRNA duplex mixture was transfected using 5 μL of Oligofectamine reagent (Invitrogen) per well according to the manufacturers instructions. Controls for the transfections included the Oligofectamine-mediated transfection of an equivalent quantity of a non-specific control duplex, a sequence that was determined to be not present in mammalian systems by BLAST analysis. The sequence of the sense strand of the non-specific randomized sequence (Scramble I Duplex, Dharmacon Research) is: 5′-CAGUCGCGUUUGCGACUGGdTdT-3′ (SEQ ID NO:3). An additional control included Oligofectamine-mediated transfection of an equivalent volume of the annealing buffer. Unless specified otherwise, cell surface levels or active caspase-3 levels were assessed on cells approximately 72 hours after transfection. Specific siRNA-mediated effects on targeted genes were confirmed by a minimum of two independent experiments.

Real time PCR analysis. RNA from cell lines was isolated from cell lines using High Pure RNA Isolation Kits (Roche). RNA samples from normal human tissues were assembled from the Human Total RNA Master Panel (Clontech) and supplemented with individual samples from First Choice Total RNA (Ambion). Depending upon the individual sample, the RNA sample from each tissue type can contain as little as one donor or represents a pooled sample from as many as 63 individuals. The quality and quantity of each RNA sample was assessed by utilization of the 2100 Bioanalyzer System (Agilent). Analysis of RNA from clinically-derived diseased tissues was outsourced to Pharmagene (Royston Hertfordshire, UK) for analysis by real time PCR. Equivalent amounts of RNA (typically 100 ng) were reverse transcribed for each condition; a consistent amount of the reaction products were utilized in the real time PCR experiments. A dilution series of a full length cDNA against L1 was utilized to generate a standard curve for quantification of the transcript. β-actin levels were monitored in samples to ensure quality of the sample was maintained over the course of several experiments. Analysis was performed on a 7900 HT real time PCR and analyzed using SDS software (Applied Biosystems). Primer and probe sets utilizing Taq chemistry (FAM/TAMRA) were used for the experiments. The following sequences were used for primers and probes against a region thought to be expressed in all known isoforms of L1:

L1/all forward primer 5′-GACTACGAGATCCACTTGTTTAAGGA-3′ (SEQ ID NO:4)

L1/all reverse primer: 5′-CTCACAAAGCCGATGAACCA-3′ (SEQ ID NO:5)

L1/all Taq probe: 5′-ATGGCACAGGCCGCGTGAGG-3′ (SEQ ID NO:6)

The following sequences were used for the detection of exon 2:

L1/exon forward primer: 5′-ATCCCCGAGGAATATGAAGGAC-3′ (SEQ ID NO:7)

L1/exon2 reverse primer: 5′-GCTCTTCCTTGGGTTTGAAGTG-3′ (SEQ ID NO:8)

L1/exon2taqprobe: 5′-TTCCCCACAGATGACATCAGCCTCAA-3′ (SEQ ID NO:9)

The following sequences were used for detection of exon 27:

L1/exon27forward primer: 5′-GGCCCGACCGATGAAAG-3′ (SEQ ID NO:10)

L1/exon27reverse primer: 5′-GCCAATGAACGAACCATCCT-3′ (SEQ ID NO:11)

L1/exon27taqprobe: 5′-TCGGCGAGTACAGGTCCCTGGAGAGTGA-3′ (SEQ ID NO:12)

Immunohistochemical Staining. Paraffin-embedded tissue array slides of normal tissues were obtained from Becton Dickinson. Arrays from ovarian, cervical and uterine diseased tissues were obtained from Innogenex, Inc. Slides were de-paraffinized at 55° C. for 10 minutes. Slides were then processed through three changes of xylene for 10 minutes each before being rehydrated through a regimen of two 5 minute treatments in 100% ethanol and one 5 minute treatment in 95% ethanol. Endogenous peroxidase activity was blocked by pre-incubation of the slide in a 3% H₂O₂ solution (LabVision) for 20 minutes. The slides were treated with Retrievagen A (Becton Dickinson) to unmask the antigenic epitope. Tissues were stained with UJ127.11 antibody at 5 μg/ml for 2 hours followed by anti-mouse streptavidin secondary antibody (LabVision) for 1 hour. Biotin-HRP was incubated on the slides for 20 minutes prior to treatment with DAB as a substrate. Samples were counterstained with Mayer's Hematoxylin (LabVision) before being processed through a dehydration regimen of two changes in 95% ethanol for 3 minutes followed by two changes of 100% ethanol for 3 minutes each. After three changes in clear xylene, cells were mounted with Permount fixing media (Fisher Scientific).

### REFERENCES

The following references, cited above, are incorporated herein in their entirety by references:
Altevogt P, and Fogel M. Diagnostic and therapeutic methods based on the L1 adhesion molecule for ovarian and endometrial tumors (Jan. 16, 2002) International Patent Application (Publ'n. No. WO 02/04952).
Balaian L B, Moehler T, and Montgomery A M (2000). Eur. J. Immunol. 30, 938-943.
Chen S, Mantei N, Dong L, and Schachner M (1999). J. Neurobiol. 38, 428-439.
Cohen N R, Taylor J S, Scott L B, Guillery R W, Soriano P, and Furley A J. (1998). Curr. Biol. 8. 26-33.
Crossin K, and Krushel L A (2000). Developmental Dynam. 218, 260-279.
Dahme M, Bartsch U, Martini R, Anliker B, Schachner M, and Mantei N. (1997) Nat Genet. 17, 346-349.
Davis J Q, and Bennett V. (1994). J. Biol. Chem. 269, 27163-27166.
Debiec H, Christensen E I, and Ronco P M (1998). J. Cell Biol. 143, 2067-2079.
Drazba J, and Lemmon V (1990). Dev Biol. 138, 82-93.
Dunn S J, Park S W, Sharma V, Raghu G, Simone J M, Tavassoli R, Young L M, Ortega M A, Pan C H, Alegre G J, Roninson I B, Lipkina G, Dayn A, and Holzmayer T A (1999). Gene Ther. 6, 130-137.
Ebeling O, Duczmal A, Aigner S, Geiger C, Schollhammer S, Kemshead J T, Moller P, Schwartz-Albiez R, and Altevogt P. (1996). Eur. J. Immunol. 26, 2508-2516.
Elbashir S M, Harborth J, Lendeckel W, Yalcin A, Weber K, and Tuschl T (2001 a). Nature, 411, 494-8.
Elbashir S M, Martinez J, Patkaniowska A, Lendeckel W, and Tuschl T (2001c). Embo J, 20, 6877-88.
Figarella-Branger D F, Durbec P L, and Rougon G N (1990). Cancer Res. 50, 6364-6370.
Fogel M, Mechtersheimer S, Huszar M, Smirnov A, Abu-Dahi A, Tilgen W, Reichrath J, Georg T, Altevogt P, and Gutwein P (2003a). Cancer Lett. 189, 237-247.
Fogel M, Gutwein P, Mechtersheimer S, Riedle S, Stoeck A, Smimov A, Edler L, Ben-Arie A, Huszar M, and Altevogt P (2003b). Lancet 362, 869-875.
Fransen E, Schrander-Stumpel C, Vits L, Coucke P, Van Camp G, and Willems P J (1994). Hum. Mol. Genet. 3, 2255-2256.
Gabrielsen T O, Brandtzaeg P, Hoel P S, and Dale I (1988). Br. J. Dermatol. 118, 59-67.
Ghosh S, Goldin E, Gordon F H, Malchow H A, Rask-Madsen J, Rutgeerts P, Vyhnalek P, Zadorova Z, Palmer T, Donoghue S; Natalizumab (2003) Pan-European Study Group N. Engl. J. Med. 348, 24-32.
Gutheil J C, Campbell T N, Pierce P R, Watkins J D, Huse W D, Bodkin D J, Cheresh D A (2000). Clin. Cancer Res. 6, 3056-61.
Hall H, Bozic D, Fauser C, and Engel J (2000). J. Neurochem. 75, 336-346.
Halliday J, Chow C W, Wallace D, and Danks D M (1986). J. Med. Genet. 23, 23-31.
Hlavin M L, and Lemmon V (1991) Genomics 11, 416-423.
Holm J, Hillenbrand R, Steuber V, Bartsch U, Moos M, Lubbert H, Montag D, and Schachner M. (1996). Eur. J. Neurosci. 8, 1613-1629.
Itoh K, Sakurai Y, Asou H, and Umeda M (2000). J. Neurosci. Res. 60, 579-586.
Jacob J, Haspel J, Kane-Goldsmith N, and Grumet M (2002). J. Neurobiol. 51, 177-189.
Jouet M, Rosenthal A, Armstrong G, MacFarlane J, Stevenson R, Paterson J, Metzenberg A, Ionasescu V, Temple K, and Kenwrick S (1994). Nature Genetics 7, 402-407.
Jouet M, Rosenthal A, and Kenwrick S (1995). Molec. Brain Res. 30, 378-380.
Kamiguchi H and Lemmon V (1998a). J. Neurosci. 18, 3749-3756.
Kamiguchi H, Hlavin M L, Lemmon V (1998b) Mol. Cell Neurosci. 12, 48-55.
Kemshead J T, Fritschy J, Garson J A, Allan P, Coakham H, Brown S, Asser U (1983). Int. J. Cancer 31, 187-195.
Kobayashi M, Miura M, Asou H, Uyemura K (1991). Biochim. Biophys. Acta. 1090, 238-240.
Kowitz A, Kadmon G, Eckert M, Schirrmacher V, Schachner M, and Altevogt P (1992). Eur. J. Immunol. 22, 1199-1205.
Kowitz A, Kadmon G, Verschueren H, Remels L, De Baetselier P, Hubbe M, Schachner M, Schirrmacher V, and Altevogt P (1993). Clin. Exp. Metastasis 11, 419-429.
Kristiansen L V, Marques F A, Soroka V, Ronn L C, Kiselyov V, Pedersen N, Berezin V, and Bock E (1999). FEBS Lett. 464, 30-34.
Lemmon V, Farr K L, and Lagenaur C (1989). Neuron 2, 1597-1603.
Mayall F G and Gibbs A R (1991). J. Pathol. 164, 47-50.
Meli M L, Carrel F, Waibel R, Amstutz H, Crompton N, Jaussi R, Moch H, Schubiger P A, and Novak-Hofer I (1999). Int. J. Cancer 83(3):401-408.
Mittleman J M and Gudkov A V (1999). Somat. Cell Mol Genet. 25, 115-128.
Miyahara R, Tanaka F, Nakagawa T, Matsuoka K, Isii K, and Wada H (2001). J. Surg. Oncol. 77(1): 49-54.
Moos M, Tacke R, Scherer H, Teplow D, Fruh K, and Schachner M (1998) Nature 334, 701-3.
Novak-Hofer I, Zimmermann K, Maecke H R, Amstutz H P, Carrel F and Schubiger P A (1993). Int. J. Cancer 53, 147-152.
Patel K, Kiely F, Phimister E, Melino G, Rathjen F, and Kemshead J T (1991). Hybridoma 10, 481-491.
Primiano T, Baig M, Maliyekkel A, Chang B D, Fellars S, Sadhu J, Axenovich S A, Holzmayer T A, and Roninson I B (2003). Cancer Cell 4, 41-53.
Reid R A and Hemperly J J (1992). J. Mol. Neurosci. 3, 127-135.
Runker A E, Bartsch U, Nave K A and Schachner M (2003). J. Neurosci. 23, 277-286.
Salceda S, Macina R A, Turner L R, Sun Y, and Liu C. Compositions and methods relating to breast specific genes and proteins (Dec. 24, 2003) International Patent Application (Publ'n. No. WO 03/106648).
Satyamoorthy K, Muyrers J, Meier F, Patel D, Herlyn M (2001). Oncogene 20, 4676-4684.
Scholl S, Beuzeboc P, and Pouillart P (2001) Ann. Oncol. 12, S81-7.
Seilheimer B, and Schachner M (1988) J. Cell Biol. 107, 341-351.
Takeda Y, Asou H, Murakami Y, Miura M, Kobayashi M, and Uyemura K (1996). J. Neurochem. 66, 2338-2349.
Thies A, Schachner M, Moll I, Berger J, Schulze H J, Brunner G, and Schumacher U (2002). Eur. J. Cancer 38, 1708-1716.
Umana P, Jean-Mairet J, Moudry R, Amstutz H and Bailey J E (1999). Nature Biotech 17, 176-180.
Voura E B, Ramjeesingh R A, Montgomery A M and Siu C (2001). Mol. Biol. Cell 12, 2699-2710.
Weller S and Gartner J (2001). Hum. Mutat. 18(1):1-12.
Yamasaki M, Thompson P and Lemmon V (1997). Neuropediatrics 28:175-178.
Yan X, Lin Y, Yang D, Shen Y, Yuan M, Zhang Z, L1 P, Xia H, L1 L, Luo D, Liu Q, Mann K, and Bader B L (2003). Blood 102, 184-191.
Yip P M and Siu C H (2001) J Neurochem 76(5):1552-1564.

Zimmermann K, Grunberg J, Honer M, Ametamey S, Schubiger P A, and Novak-Hofer 1 (2003) Nucl Med Biol 30(4):417-427.

TABLE 1

| | | Thal- | Cere- | Fetal | | |
|---|---|---|---|---|---|---|
| | Brain | amus | bellum | Brain | Placenta | Ovary |
| All L1- | 8301 | 3838 | 6231 | 44782 | 577 | 66 |
| NCAM | | | | | | |
| Exon 2 | 8151 | 4114 | 5300 | 34523 | nd* | 102 |
| Exon 27 | 5992 | 825 | 9907 | 50298 | 64 | 21 |
| | | | Mam- | | | |
| | Uterus | Testes | mary | Kidney | Heart | Lung |
| All L1- | 54 | 71 | 197 | 1698 | 15 | nd* |
| NCAM | | | | | | |
| Exon 2 | 21 | 94 | 10 | nd* | nd* | nd* |
| Exon 27 | 23 | 42 | 32 | nd* | nd* | nd* |
| | Fetal | | | | | Small |
| | Liver | Pancreas | Spleen | Stomach | Colon | Intestine |
| All L1- | nd* | 111 | 34 | 536 | 384 | 374 |
| NCAM | | | | | | |
| Exon 2 | nd* | nd* | nd* | 35 | 287 | 422 |
| Exon 27 | nd* | nd* | nd* | 13 | 219 | 176 |
| | Pros- | Salivary | | | Spinal | |
| | tate | Gland | Trachea | Thymus | Cord | Skeletal |
| All L1- | 146 | 45 | 11 | 27 | 1338 | 100 |
| NCAM | | | | | | |
| Exon 2 | 28 | nd* | 78 | 14 | 1039 | nd* |
| Exon 27 | 35 | nd* | 17 | 17 | 677 | nd* |

nd* = below detection limit

What is claimed is:

1. A method of identifying a compound that induces apoptosis in a cell, comprising:
a) contacting the cell with a putative apoptosis-inducing compound; and
b) determining whether the compound modulates the function of L1,
whereby a compound that induces apoptosis in a cell is identified.

2. The method of claim 1, wherein the determining whether the compound modulates the function of L1 comprises determining whether the compound inhibits the function of L1.

3. The method, as claimed in claim 1, wherein L1 has been validated as being involved in tumor cell growth.

4. The method, as claimed in claim 3, wherein L1 has been validated as being involved in tumor cell growth by a process comprising;
a) inhibiting the target in a cell by a method selected from the group consisting of gene knock-out, anti-sense oligonucleotide expression, use of RNAi molecules and GSE expression; and
b) assaying the cell for the ability of the cell to grow.

5. The method, as claimed in claim 1, wherein the cell is selected from tumor cell lines.

6. The method, as claimed in claim 1, wherein the step of determining is selected from the group consisting of assaying for reduced expression of L1, and assaying for reduced activity of L1.

7. The method, as claimed in claim 6, wherein the expression of L1 is measured by polymerase chain reaction.

8. The method, as claimed in claim 6, wherein the expression of L1 is measured using an antibody that specifically recognizes the target.

9. The method, as claimed in claim 6, wherein the activity of the target is measured by measuring the amount of a substrate consumed in a biochemical reaction mediated by the target.

10. The method, as claimed in claim 1, wherein the putative apoptosis-inducing compound inhibits growth of tumor cells.

11. A method for inducing apoptosis in a cell comprising inhibiting expression or activity of L1.

12. The method, as claimed in claim 11, wherein L1 has been validated as being involved in tumor cell growth.

13. The method, as claimed in claim 12, wherein L1 has been validated as being involved in tumor cell growth by a process comprising:
a) inhibiting L1 in a cell by a method selected from the group consisting of gene knock-out, anti-sense oligonucleotide expression, use of RNAi molecules and GSE expression; and
b) assaying the cell for the ability of the cell to grow.

14. The method, as claimed in claim 11, wherein the step of inhibiting is conducted by contacting a cell with an inhibitor of L1.

15. A method for the diagnosis of a tumor comprising determining the level of L1 in a patient sample, the level of the L1 being indicative of the presence of tumor cells.

16. The method as claimed in claim 15, wherein the marker level is determined by contacting a patient sample with an antibody, or a fragment thereof, that binds specifically to the marker and determining whether the anti-marker antibody or fragment thereof has bound to the marker.

17. The method as claimed in claim 15, wherein the marker level is determined using a first monoclonal antibody that binds specifically to the marker and a second antibody that binds to the first antibody.

18. The method as claimed in claim 15, wherein the bodily fluid is immobilized.

19. The method as claimed in claim 15, wherein the method is used to determine the prognosis for cancer in the patient.

20. The method as claimed in claim 15, wherein the method is used to determine the susceptibility of the patient to a therapeutic treatment.
